(19) United States
(12) Patent Application Publication
(10) Pub. No.: US 2012/0016320 A1
(43) Pub. Date: Jan. 19, 2012

(54) Method and Device for Liquid Delivery

(76) Inventor: Michael T. Lin, Sherman Oaks, CA (US)

(21) Appl. No.: 12/835,324

(22) Filed: Jul. 13, 2010

(51) Int. Cl. A61M 35/00; B67D 3/00

(52) U.S. Cl. 604/290; 222/192; 222/1; 604/310

(57) ABSTRACT

A liquid delivery device is provided which allows for delivery of precise, small amounts of a target liquid, such as cantharadin, glycolic acid, phenol, trichloroacetic acid, or similar liquids to a specific, discrete target location, such as an area of the skin to treat conditions including, but not limited to: molluscum contagiosum, verrucous vulgaris (warts), verrucous plana (flat warts), acne scars, actinic keratosis, acrochordon (skin tags), seborrheic keratosis, dermatosis papulosa nigra, milia and lentigo (sun spots). The liquid delivery device allows for delivery of the liquid to the target area without delivering the liquid to any part of the area surrounding the precise target location, thereby eliminating unnecessary irritation such as blistering, pain, temporary or permanent tissue damage and/or scarring for the user. The liquid delivery device strongly adheres to the target liquid, eliminating spilling and the resultant danger to the user and eliminating economic waste. In one implementation, the device comprises a small, hollow tube, the material and diameter of which allow uptake and retention of a target liquid by taking advantage of capillary action and surface tension.

This generally relates to devices used in dermatology, cosmetology, ophthalmology and optometry and more particularly to devices for the precision administration of medication or cosmetics in exact amounts to specific locations.

### BACKGROUND

The practices of dermatology, cosmetology, ophthalmology, optometry, and other like services often require the transfer and/or application of liquid medications or cosmetics, including aqueous solutions, true solutions, oils, solvents, emulsions, creams, ointments, lotions, suspensions, pastes, jellies, syrups, balms, or any other similar substance to an exact and/or small location on the skin, eye, or other part of the human or animal body to treat conditions including, but not limited to: molluscum contagiosum, verrucous vulgaris (warts), verrucous plana (flat warts), acne scars, actinic keratosis, acrochordon (skin tags), seborrheic keratosis, dermatosis papulosa nigra, milia and lentigo (sun spots). This is normally accomplished by a number of methods including small brushes or solid rod applicators including glass rods, toothpicks, and cotton swabs such as a Q-Tip.

A typical pipette includes a slender pipe or tube that is used to measure and/or transfer small quantities of a target liquid or gas from one location to another. The most common type of pipette includes a small plastic tube that widens into a bulb at the end. The bulb is squeezed to expel air, and then the tip is placed into a target liquid and the bulb is slowly released, drawing the liquid up into the pipette. Then, the liquid is dispensed through forcible expulsion by squeezing the bulb. Another common embodiment is the mechanical pipette, which allows the user to mechanically select a desired volume before depressing a plunger, dipping the pipette tip into a target liquid, and slowly releasing the plunger to draw the pre-set amount of the liquid up into the pipette tip. Then, the plunger is depressed, dispensing the liquid through forcible expulsion. This forcible expulsion of the liquid is fast and useful in applications where the liquid is merely being measured and/or relocated, but imprecise in applications where a small and distinct amount of the liquid must be carefully delivered to a precise target location.

A typical brush applicator includes a rod-shaped handle with a mass of bristles attached to the distal end. The bristles of the brush are dipped into a target liquid, and then the bristles with the liquid adhering to them are brushed across the target surface the liquid is to be applied to, transferring the liquid from the bristles to the surface. This brushing causes an inaccurate transfer of the liquid over most of the surface the bristles touch, useful in applications where the liquid is being dispersed over a wide area, but imprecise in applications where a small and distinct amount of the liquid must be delivered to a precise target location.

A typical rod applicator comprises a glass rod, wooden or plastic toothpick, or a cotton swab such as a Q-Tip. The distal end of the rod is dipped into a target liquid, and then the distal end of the rod with the liquid adhering to it is dabbed onto the target location, transferring the liquid from the distal end of the rod to the location. This device allows more accurate application than brush applicators, but does not allow for accurate measurement of the amount of the liquid dispensed.

Conventional application of dermatologic, cosmetic, ophthalmologic and optometric liquids with any of these typical applicators has several drawbacks. First, many of the target liquids applied in these practices are corrosive or otherwise irritating to the human or animal skin. None of these conventional application devices allow for the precise application of the liquids to the precise target location without also spreading the liquids to the surrounding areas of skin, causing unnecessary irritation and patient discomfort. Such potential negative side effects of inaccurate and imprecise volumetric and locational application of liquid medications and/or cosmetics may include blistering, pain, temporary or permanent tissue damage and/or scarring. Second, brushes and rods do not allow precise measurement of the target liquid volume collected or transferred. Third, brushes and rods loosely adhere to the target liquid, allowing for dripping during transfer. This creates two distinct problems. One is the potential for the drops to come in contact with the skin of the physician or other health professional, or the skin of the patient or user outside the target area, causing unnecessary irritation and discomfort including the aforementioned blistering, pain, temporary or permanent tissue damage and/or scarring. The other is the economic disadvantages inherent in inefficient use of these target liquids given the often high costs of the liquids.

Accordingly, there is a desire for a liquid delivery device which allows for delivery of precise, small amounts of a target liquid to a specific, discrete target location.

### SUMMARY

In accordance with the methods and devices consistent with the present invention, a liquid delivery device is provided for storing and applying a liquid to an application site, comprising a hollow cylinder configured to apply the liquid to the application site. The hollow cylinder further comprises an open upper end and an open lower end, and an inner diameter less than 2.0 millimeters. The inner diameter of the hollow cylinder, a length of the hollow cylinder, and a material of which the hollow cylinder is composed, permit capillary action to draw the liquid into the hollow cylinder for subsequent application to the application site, and to dispense the liquid on the application site.

In one implementation, a method of applying liquid to an application site is provided, comprising touching a liquid delivery device to a liquid to draw the liquid into the device. The liquid delivery device is configured to store and apply a liquid medication to an application site and comprises a hollow cylinder configured to apply the liquid to the application site. The hollow cylinder comprises an open upper end and an open lower end; and an inner diameter less than 2.1 millimeters. The inner diameter of the hollow cylinder, a length of the hollow cylinder, and a material of which the hollow cylinder is composed, permit capillary action to draw the liquid into the hollow cylinder for subsequent application to the application site, and to dispense the liquid on the application site. The method further comprises touching the liquid delivery device to the application site to apply the liquid.

### BRIEF DESCRIPTION OF THE DRAWINGS

FIG. 1 depicts a side view of a capillary tube applicator in accordance with methods and devices consistent with the present invention.

FIG. 2 depicts a side view of an alternative embodiment of a capillary tube applicator in accordance with methods and devices consistent with the present invention.

FIG. 3 depicts a side view of an alternative embodiment of a capillary tube applicator in accordance with methods and devices consistent with the present invention.

FIG. 4 depicts a side view of an alternative embodiment of a capillary tube applicator in accordance with methods and devices consistent with the present invention.

FIG. 5 illustrates steps in a method for applying medication using a capillary tube applicator in accordance with the present invention.

### DETAILED DESCRIPTION

Methods and systems in accordance with the present invention provide a liquid delivery device which allows for delivery of precise, small amounts of a target liquid, such as cantharadin, glycolic acid, phenol, trichloroacetic acid or similar liquids to a specific, discrete target location, such as an area of the skin to treat conditions including, but not limited to: molluscum contagiosum, verrucous vulgaris (warts), verrucous plana (flat warts), acne scars, actinic keratosis, acrochordon (skin tags), seborrheic keratosis, dermatosis papulosa nigra, milia and lentigo (sun spots). The liquid delivery device allows for delivery of the liquid to the target area without delivering the liquid to the area surrounding the precise target location, thereby eliminating unnecessary irritation, such as blistering, pain, temporary or permanent tissue damage and/or scarring for the patient or user. The liquid delivery device strongly adheres to the target liquid, eliminating spilling and the resultant danger to the physician, other practitioner, patient, or user and eliminating economic waste. Generally, the device comprises a small, hollow tube, the material and diameter of which allow uptake and retention of a target liquid by taking advantage of capillary action and surface tension. Exemplary diameters for the interior hollow portion of the tube include 0.1 mm and 2.0 mm, or diameters in between, and an exemplary length of the tube is 7.5 cm. In one implementation, diameters for the interior hollow portion of the tube include 0.7 mm and 1.1 mm, or diameters in between. Other diameters and lengths may also be used.

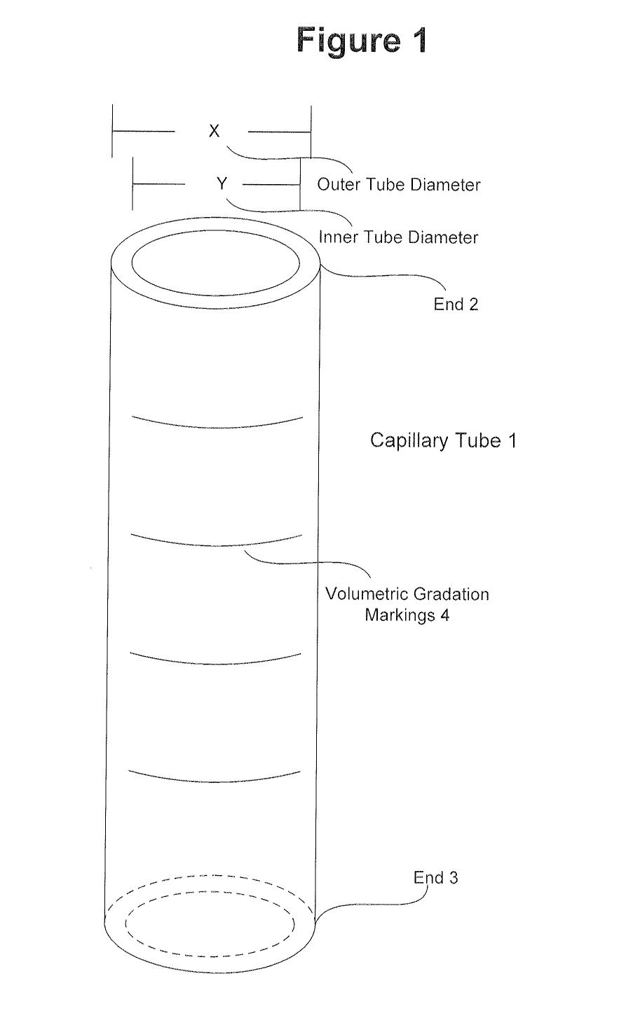

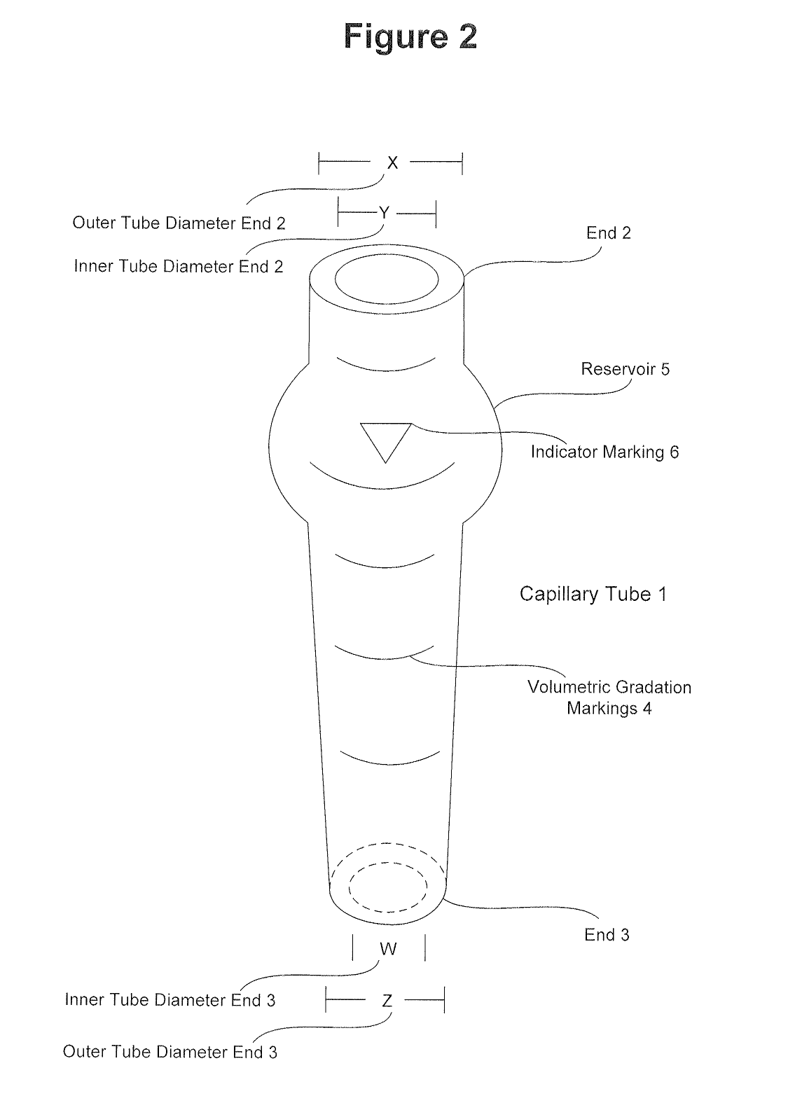

FIG. 1 illustrates an exemplary embodiment of a capillary tube applicator made in accordance with methods and systems consistent with the present invention. In one implementation, capillary tube 1 is hollow throughout and open on both end 2 and end 3. The material used to construct capillary tube 1 may vary depending on the target liquid to be used in conjunction with the specific tube, and may comprise any material that is not corroded by the liquid, including but not limited to glass, metal, plastic, ceramic, clay, wood, or any other suitable material. In the exemplary embodiment shown in FIG. 1, both end 2 and end 3 have the same inner tube diameter Y and outer tube diameter X. In other exemplary embodiments depicted in FIGS. 2-4, end 2 and end 3 have differing inner tube diameters and outer tube diameters. A physician, other practitioner or user places either end 2 or end 3 into a target liquid. If capillary tube 1 is sufficiently narrow and the cohesion of the liquid and adhesion of the liquid to the material of construction of capillary tube 1 are sufficiently strong, capillary action draws the liquid up into the tube, to a specific height.

Capillary action is the tendency of a liquid to rise in narrow tubes. It is a result of cohesion within a liquid and adhesion between a liquid and a solid forming the void, such as the material of construction of capillary tube 1. Cohesion is the mutual attractive force that exists between like molecules of a particular liquid. Cohesion produces surface tension. Adhesion is the attractive force that exists between two unlike materials, such as the target liquid and the material of construction of capillary tube 1. A narrower tube, greater cohesion and surface tension within a liquid, an increased ratio of adhesion to cohesion between a liquid and the solid forming the void, and a lower density liquid all cause the liquid to rise to a greater height within capillary tube 1.

The physician, other practitioner or user may observe the precise volume of liquid uptake using the volumetric gradation markings 4 located on the outside of the capillary tube 1. Once capillary action has moved the liquid into capillary tube 1, the physician, other practitioner or user can remove the end 2 or end 3 from the liquid, and the liquid will adhere to the inside of capillary tube 1 without dripping because of surface tension. To release the liquid from capillary tube 1, the physician, other practitioner or user physically touches the end of capillary tube 1 which was initially submerged in the liquid to the application site, such as a specific area of the skin. This breaks the surface tension of the liquid inside the tube, causing the liquid to flow out of the tube until the physician, other practitioner or user breaks the physical contact between the end of capillary tube 1 and the application site. The physician, other practitioner or user may calculate the precise volume of liquid applied to the application site using the volumetric gradation markings 4. Additionally, the tube may be different colors, such as yellow, to enhance visualization of the liquid in the tube.

FIG. 2 depicts another exemplary embodiment of a capillary tube applicator in accordance with methods and devices consistent with the present invention wherein end 2 has an outer tube diameter X and an inner tube diameter Y while end 3 has an outer tube diameter Z, which is less than X and an inner tube diameter W, which is less than Y. This disparity in size allows end 2 and end 3 to be used alternately in the application of different target liquids or in the application of different volumes of the same liquid. This is in part because one of the variables affecting the height a column of liquid rises through capillary action is r, the radius of the capillary, which is equal to ½ the diameter of the capillary. In this exemplary embodiment, capillary tube 1 includes an indicator marking 6 to indicate the smaller diameter end 3. In one implementation, the difference in diameters between end 2 and end 3 in this embodiment is on an order of millimeters or less, a measurement scale often imperceptible to the human eye. Thus, this marking allows the physician, other practitioner or user to discern the end 3 of smaller diameter. In this exemplary embodiment, capillary tube 1 includes a reservoir 5 which is located at a distance U from end 2 and a distance V from end 3.

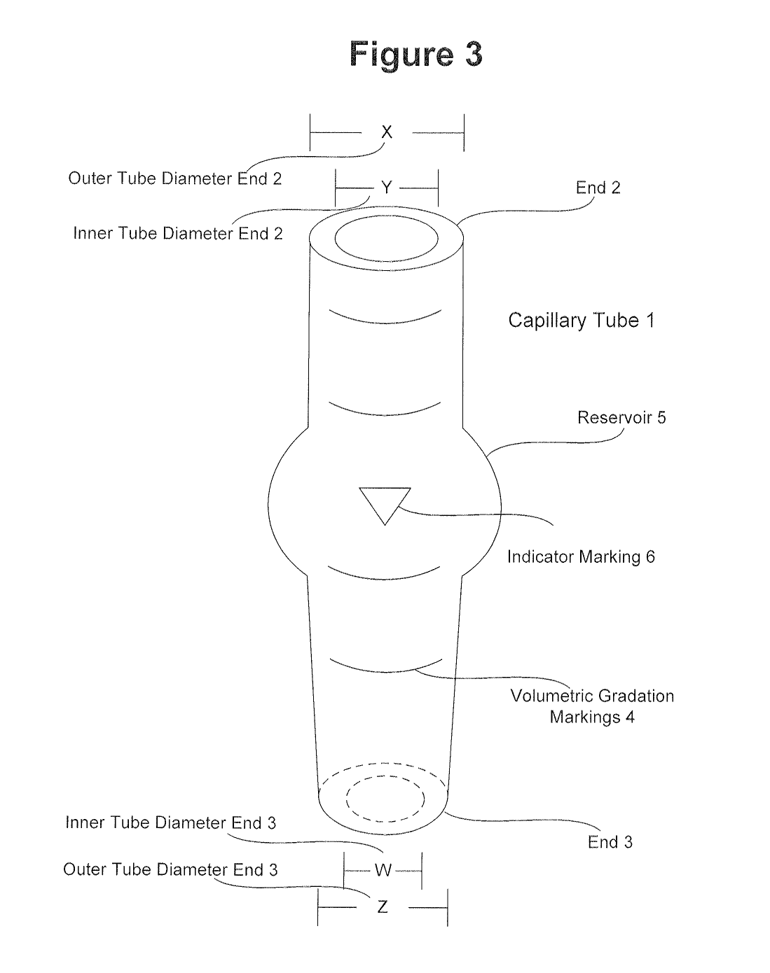

FIG. 3 depicts another exemplary embodiment of a capillary tube applicator in accordance with methods and devices consistent with the present invention wherein reservoir 5 is located at the same distance T from both end 2 and end 3. In the exemplary embodiment shown in FIG. 3, reservoir 5 is equidistant or roughly equidistant from end 2 and end 3. The purpose of reservoir 5 is twofold. First, reservoir 5 allows the physician, other practitioner or user to uptake a large quantity of liquid upon a single submersion of end 2 or end 3 in the liquid, allowing for either one large application or multiple small applications without requiring re-submergence of end 2 or end 3 to collect more liquid in capillary tube 1. Second, the reservoir provides more head space between the top surface of the liquid and the proximal end of capillary tube 1, ameliorating the possibility of the liquid overflowing and causing injury to the physician or other practitioner or to the patient.

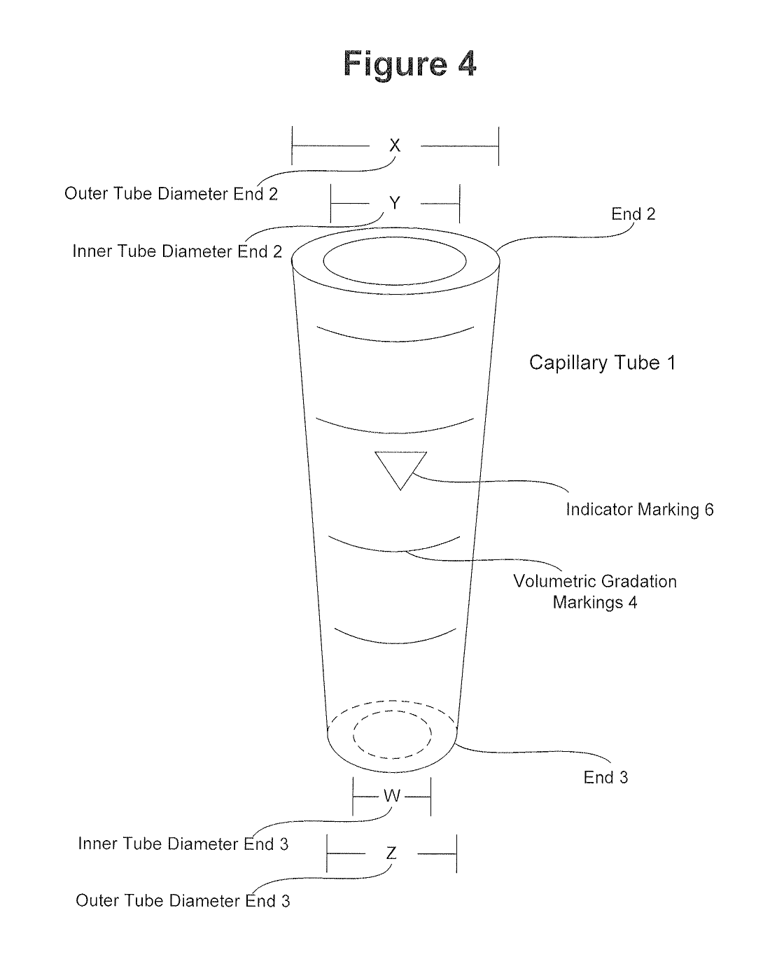

FIG. 4 depicts another exemplary embodiment of a capillary tube applicator in accordance with methods and devices consistent with the present invention wherein reservoir 5 is not present, and wherein end 2 has an outer tube diameter X and an inner tube diameter Y while end 3 has an outer tube diameter Z, which is less than X and an inner tube diameter W, which is less than Y. This embodiment captures the benefits associated with having end 2 and end 3 of disparate sizes as in the embodiment shown in FIGS. 2 and 3 without the necessity of reservoir 5.

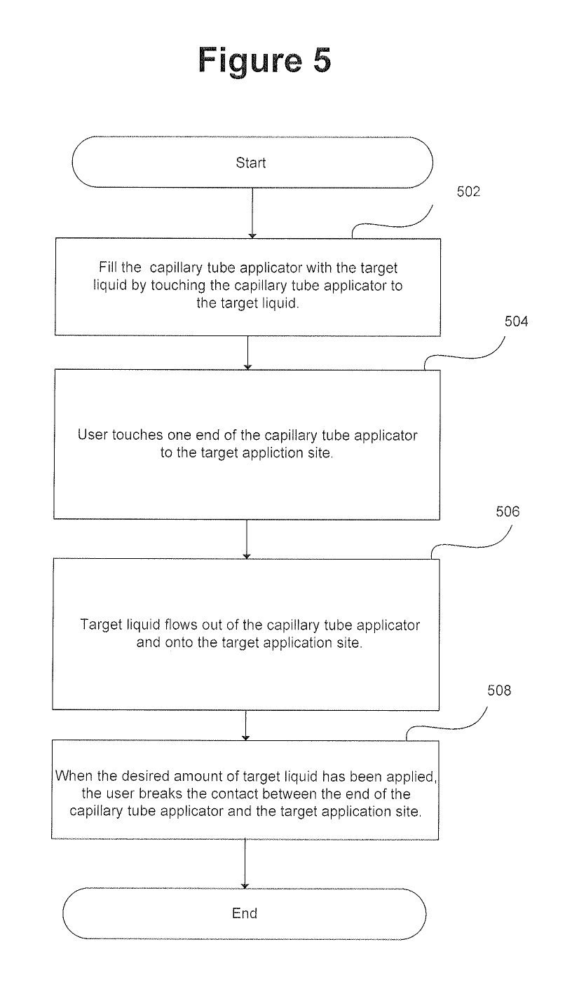

FIG. 5 illustrates a flowchart depicting steps in a method for using a capillary tube applicator in accordance with the present invention. First, the user fills the capillary tube applicator 1 with the specific amount of the target liquid by touching the capillary tube applicator 1 to the target liquid until it has reached the target amount (step 502). As mentioned, examples of this target liquid include cantharadin, glycolic acid, phenol, or trichloroacetic acid. Next, the user touches one end of the capillary tube applicator 1 to the target application site (step 504), such as a specific area of the skin or a wart, for example. The target liquid then leaves the capillary tube applicator 1 and applies to the application site (step 506). When the proper amount has been applied, the user removes the end of the capillary tube applicator 1 from the target site (step 508).

The foregoing description of various embodiments provides illustration and description, but is not intended to be exhaustive or to limit the invention to the precise form disclosed. Modifications and variations are possible in light of the above teachings or may be acquired from practice in accordance with the present invention. It is to be understood that the invention is intended to cover various modifications and equivalent arrangements included within the spirit and scope of the appended claims.

What is claimed is:

1. A liquid delivery device for storing and applying a liquid to an application site, comprising:
a hollow cylinder configured to apply the liquid to the application site, comprising:
an open upper end and an open lower end; and
an inner diameter less than 2.1 millimeters;
wherein the inner diameter of the hollow cylinder, a length of the hollow cylinder, and a material of which the hollow cylinder is composed, permit capillary action to draw the liquid into the hollow cylinder for subsequent application to the application site, and to dispense the liquid on the application site.

2. The liquid delivery device of claim 1, wherein the material is one of: (1) glass, (2) metal, (3) wood, (4) plastic, (5) clay, and (6) ceramic.

3. The liquid delivery device of claim 1, wherein the cylinder comprises the liquid, and wherein the liquid is a medication.

4. The liquid delivery device of claim 3, wherein the cylinder comprises the liquid, wherein the liquid is cantharidin.

5. The liquid delivery device of claim 3, wherein the liquid is one of: (1) glycolic acid, (2) phenol, and (3) trichloroacetic acid.

6. The liquid delivery device of claim 1, wherein the inner diameter is between 0.7 millimeters and 1.1 millimeters.

7. The liquid delivery device of claim 1, wherein the inner diameter is greater than 0.1 millimeters.

8. The liquid delivery device of claim 1, wherein the inner diameter is 0.7 millimeters.

9. The liquid delivery device of claim 1, wherein the length of the hollow cylinder is 7.5 centimeters.

10. The liquid delivery device of claim 1, wherein the application site is on skin.

11. The liquid delivery device of claim 1, wherein the open upper end is the same size as the open lower end.

12. The liquid delivery device of claim 1, wherein the open upper end is wider than the open lower end.

13. The liquid delivery device of claim 1, further comprising a reservoir having a width wider than the inner diameter of an upper portion of the hollow cylinder and a lower portion of the hollow cylinder.

14. A method of applying liquid to an application site, comprising:
touching a liquid delivery device to a liquid to draw the liquid into the device;
wherein the liquid delivery device is configured to store and apply a liquid medication to an application site and comprises:
a hollow cylinder configured to apply the liquid to the application site, comprising:
an open upper end and an open lower end; and
an inner diameter less than 2.1 millimeters;
wherein the inner diameter of the hollow cylinder, a length of the hollow cylinder, and a material of which the hollow cylinder is composed, permit capillary action to draw the liquid into the hollow cylinder for subsequent application to the application site, and to dispense the liquid on the application site; and
touching the liquid delivery device to the application site to apply the liquid.

15. The method of claim 14, wherein touching the liquid device to the liquid further comprises:
stopping the touching of the liquid delivery device to the liquid when a desired amount of liquid has entered the liquid delivery device.

16. The method of claim 14, wherein touching the liquid device to the application side further comprises:
stopping the touching of the liquid delivery device to the application site when a desired amount of liquid is put on the application site.

17. The method of claim 14, wherein the material is one of: (1) glass, (2) metal, (3) wood, (4) plastic, (5) clay, and (6) ceramic.

18. The method of claim 14, wherein the cylinder comprises the liquid, and wherein the liquid is a medication.

19. The method of claim 18, wherein the cylinder comprises the liquid, and wherein the liquid is cantharidin.

20. The method of claim 18, wherein the cylinder comprises the liquid, and wherein the liquid is one of: (1) glycolic acid, (2) phenol, and (3) trichloroacetic acid.

21. The method of claim 14, wherein the inner diameter is between 0.7 millimeters and 1.1 millimeters.

22. The method of claim 14, wherein the inner diameter is greater than 0.1 millimeters.

23. The method of claim 14, wherein the inner diameter is 0.7 millimeters.

24. The method of claim 14, wherein the length of the hollow cylinder is 7.5 centimeters.

25. The method of claim 14, applying the liquid to skin.

26. The method of claim 14, wherein the open upper end is the same size as the open lower end.

27. The method of claim 14, wherein the open upper end is wider than the open lower end.

28. The method of claim 14, wherein the liquid delivery device further comprises a reservoir having a width wider than the inner diameter of an upper portion of the hollow cylinder and a lower portion of the hollow cylinder.
